(19) United States
(12) Patent Application Publication
(10) Pub. No.: US 2014/0295011 A1
(43) Pub. Date: Oct. 2, 2014

(54) Composition Containing Triticum Aestivum Lamarck Leaf Extract or Fraction Thereof as Active Ingredient

(71) Applicant: GENEMATRIX INC., Seongnam-si, Gyeonggi-do (KR)

(72) Inventors: Young-Mi Lee, Iksan-si Jeonbuk (KR); Dae-Ki Kim, Jeonju-si (KR); Joo-Won Suh, Yongin-si (KR); Sun-Hee Lee, Jeonju-si (KR)

(73) Assignee: GENEMATRIX INC., Seongnam-si, Gyeonggi-do (KR)

(21) Appl. No.: 14/353,373

(22) PCT Filed: Oct. 26, 2012
(86) PCT No.: PCT/KR2012/008849

§ 371 Date: Apr. 22, 2014

(30) Foreign Application Priority Data
Oct. 27, 2011 (KR) 10-2011-0110385

(51) Int. Cl. A61K 36/899; A23L 1/30

(52) U.S. Cl. CPC A61K 36/899; A23L 1/3002
USPC 424/774

(57) ABSTRACT

The present invention relates to a pharmaceutical composition for prevention and treatment of hyperlipidemia or fatty liver disease, the composition containing dichloromethane fractions as an active ingredient amongst fractions obtained by fractionating the alcohol extract of Triticum aestivum Lamarck leaves in an order of dichloromethane, ethyl acetate, and butanol.

### TECHNICAL FIELD

The present invention relates to a Triticum aestivum Lamarck leaf extract or a composition containing a fraction thereof as an active ingredient.

### BACKGROUND ART

Non-alcoholic fatty liver disease is the most common liver disease and is defined as a disease where the content of triglycerides exceeds 5% of the weight of liver. In particular, obesity and type 2-diabetes have been drawing much public interest because they are known to be the most risky factors for inducing non-alcoholic fatty liver disease. Obesity is a phenomenon in which excessive body fat accumulates due to the imbalance between energy intake and energy consumption in the body causing an increase in the number and the size of adipocytes. Obesity is a risk factor which not only changes the body shape due to the accumulation of fat in the abdomen and intestines but is also associated with the increase in various diseases such as insulin resistance, dyslipidemia, hypertension, diabetes, arteriosclerosis, etc.

Preadipocytes are continuously differentiated into adipocytes through the life of a living organism. Insulin is the most well-known hormone which serves as a signal to induce the differentiation of preadipocytes into adipocytes, and plays a crucial role in the metabolic control of adipocytes. Insulin increases the absorption of saccharides and synthesis of triglycerides and stores energy in the form of a lipid. Lipid droplets in the adipocytes are known to play an important role in the metabolism and regulation of lipids, and regulate the synthesis of triglycerides and the accumulation of lipids in the differentiated preadipocytes. As such, adipogenesis occurs via a sophisticated process including changes in cell shape, hormone sensitivity, gene expression, protein expression, and is also associated with metabolic diseases such as obesity, diabetes, etc. Adipocytes have been reported to be formed through a process including the increase in the expression of transcription factors such as CCAAT/enhancer binding proteins (C/EBPs) and peroxidase proliferator-activated receptor-gamma (PPAR-g), induction of fatty acid synthase (FAS), and regulation of lipid accumulation within the apocytes.

Triticum aestivum Lamarck is a gramineous plant, which is mostly cultivated in temperate regions, and is also a crop with high production being highly favored as a staple food in the world. The juice of Triticum aestivum Lamarck has been known to be effective in the treatment of cancers by inducing apoptosis of cancer cells, and also known to inhibit the activity of 7,2-dimethylbenz(a)anthracene, a carcinogenic substance. Additionally, Triticum aestivum Lamarck has been reported to have various pharmacological efficacies such as anti-inflammation and antioxidization activities. However, there have been no reports on the relationship between the Triticum aestivum Lamarck extract and the lipid synthesis by adipocytes.

### PRIOR ART DOCUMENTS

### Non-Patent Documents

(Non-patent Document 0001) Lai, C. N.: Chlorophyll: The active factor in wheat sprout extract inhibiting the metabolic activation of carcinogens in vitro. Nutri. Cancer 3, 19-21 (1979).

(Non-patent Document 0002) Ben-Arye, E., Goldin, E., Wengrower, D., Stamper, A., Kohn, R. and Berry, E.: Wheat grass juice in the treatment of active distal ulcerative colitis: a randomized double-blind placebo-controlled trial. Scand. J. Gastroenterol 37, 444-449 (2002).

(Non-patent Document 0003) Watzl, B.: Anti-inflammatory effects of plant-based foods and of their constituents. Int. J. Vitam. Nutr. Res. 78, 293-298 (2008).

### DISCLOSURE

### Technical Problem

In an aspect of the present invention, there is provided a composition containing a Triticum aestivum Lamarck leaf extract or a fraction thereof as an active ingredient.

In another aspect of the present invention, there is provided a health food composition for the prevention or improvement of obesity including an extract of Triticum aestivum Lamarck leaves or a fraction thereof as an active ingredient.

### Technical Solution

In an exemplary embodiment of the present invention, there is provided a pharmaceutical composition for the prevention or treatment of hyperlipidemia and fatty liver disease including a dichloromethane fraction as an active ingredient out of fractions obtained by sequential systemic fractionation of an alcohol extract of Triticum aestivum Lamarck leaves with dichloromethane, ethyl acetate, and butanol in that order

In an exemplary embodiment of the present invention, the alcohol extract may be a 50% ethanol extract.

In an exemplary embodiment of the present invention, the dichloromethane fraction may inhibit the expression of protein PPARγ.

In an exemplary embodiment of the present invention, the dichloromethane fraction may inhibit the expression of protein FAS.

In an exemplary embodiment of the present invention, there is provided a health food composition for the prevention or improvement of obesity including a dichloromethane fraction as an active ingredient, out of fractions obtained by sequential systemic fractionation of an alcohol extract of Triticum aestivum Lamarck leaves with dichloromethane, ethyl acetate, and butanol in that order.

Given an extract of Triticum aestivum Lamarck leaves or a fraction thereof as an active ingredient, or a fraction thereof, a pharmaceutical composition may include at least one different active ingredient exhibiting the same or similar functions according to an exemplary embodiment of the present invention. The extract of Triticum aestivum Lamarck leaves or a fraction thereof may be clinically administered via an oral or parenteral route, and may be used in the form of a general pharmaceutical formulation.

In other words, the extract of Triticum aestivum Lamarck leaves or a fraction thereof may be clinically administered as various oral or parenteral dosage forms. In this regard, the pharmaceutical composition of the present invention may be formulated in combination with a diluent or excipient such as a filler, a thickener, a binder, a humectant, a disintegrant, a surfactant, etc. Solid preparations intended for oral administration may be in the form of tablets, pills, powders, granules, capsules, and the like. In regards to these solid agents, the extract of Triticum aestivum Lamarck leaves of the present invention is formulated in combination with at least one excipient such as starch, calcium carbonate, sucrose, lactose, or gelatin. In addition to a simple excipient, a lubricant such as magnesium stearate, talc, etc. may be used. Among liquid preparations intended for oral administration are suspensions, internal use solutions, emulsion, syrups, and the like. Plus a simple diluent such as water or liquid paraffin, various excipients, such as humectants, sweeteners, aromatics, preservatives, and the like may be contained in the liquid preparations. Also, the pharmaceutical composition of the present invention may be in a parenteral dosage form such as sterile aqueous solutions, non-aqueous solvents, suspensions, emulsions, lyophilizates, suppositories, and the like. Injectable propylene glycol, polyethylene glycol, vegetable oils such as olive oil, and esters such as ethyl oleate may be suitable for the non-aqueous solvents and suspensions. The basic materials of suppositories include Witepsol, macrogol, Tween 61, cacao butter, laurin butter, and glycerogelatin.

The dosage of the extract of the present invention or a fraction thereof may vary greatly depending on the patient's weight, age, health conditions, sex, and diet, the time of administration, the route of administration, the excretion rate, the severity of disease, etc. The extract of Triticum aestivum Lamarck leaves or a fraction thereof may be administered at a dose of from 0.1 to 100 mg/kg/day, preferably at a dose of from 30 to 80 mg/kg/day, and more preferably at a dose of from 50 to 60 mg/kg. The active ingredient according to the present invention may be administered in a single dosage or may be divided into 6 dosages per day.

The extract of the present invention may be used singly or in combination with surgery, hormone therapy, pharmacological therapy and biological response modulators.

In an exemplary embodiment of the present invention, there is provided a method for the treatment of hyperlipedemia including administering a pharmaceutically effective amount of the extract of Triticum aestivum Lamarck leaves or a fraction thereofor a fraction thereof to a subject.

In an exemplary embodiment of the present invention, there is provided a method for the treatment of a fatty liver disease including administering a pharmaceutically effective amount of the extract of Triticum aestivum Lamarck leaves or a fraction thereof to a subject.

In an exemplary embodiment of the present invention, there is provided a method for the prevention of hyperlipedemia including administering a pharmaceutically effective amount of the extract of Triticum aestivum Lamarck leaves or a fraction thereof to a subject.

In an exemplary embodiment of the present invention, there is provided a method for the prevention of a fatty liver disease including administering a pharmaceutically effective amount of the extract of Triticum aestivum Lamarck leaves or a fraction thereof to a subject.

In particular, the pharmaceutically effective amount may be preferably about from 0.1 to 500 mg/kg, more preferably about from 1 to 100 mg/kg, but is not limited thereto. The dosage may vary depending on the patient's weight, age, health conditions, sex, and diet, the time of administration, the route of administration, the excretion rate, the severity of disease, etc.

In an exemplary embodiment of the present invention, there is provided a health food composition for the prevention or improvement of obesity the extract of Triticum aestivum Lamarck leaves or a fraction thereof as an active ingredient.

When used as a food additive, the extract of Triticum aestivum Lamarck leaves or a fraction thereof may be added as they are, or in combination with other food components using a typical method. Its amount in the mixture may be properly determined depending on the purpose of use. (e.g., prevention, health or therapeutic treatment). Generally, when used for the preparation of foods or beverages, the composition of the present invention may be added in an amount of 15 parts by weight or less based on the total weight of the health food, and preferably in an amount of 10 parts by weight or less. In the case where the active ingredient is applied to health foods which are designed to be taken for a long term period for improving health or hygienic purpose, or to control health conditions, its content may be below the above-mentioned range. Since the active ingredient does not raise any safety issues, its amount may be beyond the above range as well.

No particular limitations are imposed on the kind of foods to which the active ingredient can be applied. Examples of the foods include meat, sausages, bread, chocolate, candies, snacks, confectionaries, pizza, ramen and other noodles, gums, dairy products including ice creams, soups, beverages, teas, drinks, alcoholic beverages, vitamin tablets, etc., but are not limited thereto. All usually accepted health foods may contain the active ingredient according to the present invention.

The health food composition may take a health drink form. The health drink according to the present invention may further contain various fragrant or natural carbohydrates. Examples of the natural carbohydrates include monosaccharides such as glucose and fructose; disaccharide such as maltose and sucrose; polysaccharides such as dextrin and cyclodextrin; and sugar alcohols such as xylitol, sorbitol, erythritol, etc. Also, sweeteners, e.g., natural sweeteners such as thaumatin and a stevia extract, or synthetic sweeteners such as saccharin and aspartame, may be added to the health food to which the active ingredient of the present invention is applied. The natural carbohydrate may be used in an amount of about 0.01˜0.04 g based on 100 mL of the composition of the present invention, and preferably in an amount of about 0.02˜0.03 g.

In addition, the health food composition of the present invention may contain various nutrients, vitamins, minerals (electrolytes), flavors, colorants, pectic acid and salts thereof, alginic acid and salts thereof, organic acids, protective colloidal thickeners, pH modifiers, stabilizers, antiseptics, glycerin, alcohols, and carbonating agents used in carbonated beverages. Moreover, the composition of the present invention can contain fruit flesh for preparing natural fruit juices, fruit beverages and vegetable beverages. These ingredients may be used individually or in combination. The ratio of these additives is not important, but is generally selected in a range of 0.01 to 0.1 parts by weight per 100 parts by weight of the composition of the present invention. In the present invention, the term “obesity” refers to a state where fat tissues have been abnormally increased in the body whether it is due to a genetic reason or an environmental reason, which includes classifications such as obesity (where body mass index (BMI) is 30 or above) or overweight (where BMI is 25-30) according to BMI.

In the present invention, the term “active ingredient” refers to an ingredient which may exhibit its intended purpose alone or in combination with an excipient which does not exhibit any activity itself.

In the present invention, the term “obesity improvement” refers to a reduction of body fat and/or body weight including the prevention and treatment of obesity.

In an exemplary embodiment of the present invention, the composition of the present invention for the improvement of obesity may include a random amount (an effective amount) of the active ingredients depending on the use, type of formulations, mixing purposes, as long as they may exhibit the obesity improving activities, and the conventional effective amount may be from about 0.001 wt % to about 99.990 wt %. In the present invention, the term “effective amount” may refer to the amount of the active ingredients that may induce the improvement in obesity. The effective amount may be experimentally determined within the conventional range by a person skilled in the art.

In an exemplary embodiment of the present invention, the composition of the present invention may be applied (transcribed) to mammals including humans, preferably humans.

### Advantageous Effects

As described above, the pharmaceutical composition of the present invention is effective in the prevention or treatment of hyperlipidemia and fatty liver disease.

Furthermore, the health food composition of the present invention is effective in the prevention or treatment of obesity.

### DESCRIPTION OF DRAWINGS

FIG. 1 is a flowchart for preparing a Triticum aestivum Lamarck leaf extract and fractions thereof as an active ingredient.

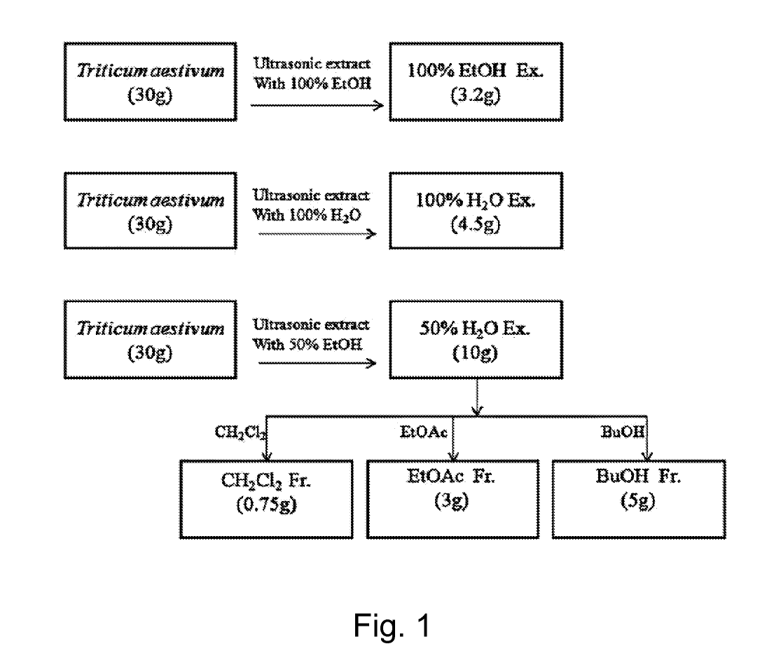

FIG. 2 is a graph illustrating the cell viability of 3T3-L1 preadipocytes treated with varying concentrations of (a)H₂O, (b)50% EtOH and (c)100% EtOH extracts of Triticum aestivum Lamarck leaves.

FIG. 3 is a graph illustrating the effect of (a)H₂O, (b)50% EtOH and (c)100% EtOH extracts of Triticum aestivum Lamarck leaves on the fat accumulation of the 3T3-L1 adipocytes.

FIG. 4 is a graph illustrating the effect of CH₂Cl₂, EtOAc, and BuOH fractions of 50% EtOH extract Triticum aestivum Lamarck leaves on the fat accumulation of the 3T3-L1 adipocytes.

FIG. 5 shows pictures illustrating the effect of the CH₂Cl₂ fraction of 50% EtOH extract of Triticum aestivum Lamarck leaves on the differentiation of the 3T3-L1 preadipocytes.

FIG. 6 is a graph illustrating the effect of the CH₂Cl₂ fraction of 50% EtOH extract of Triticum aestivum Lamarck leaves on the triglyceride content of the differentiated 3T3-L1 adipocytes.

FIG. 7 shows results of western blot analysis confirming the effect of the CH₂Cl₂ fraction of 50% EtOH extract of Triticum aestivum Lamarck leaves on the expression of fatty acid synthase (FAS) and peroxisome proliferator-activated receptor-gamma (PPARg) in the 3T3-L1 cells.

### MODE FOR INVENTION

Hereinafter reference will now be made in detail to various embodiments of the present invention, examples of which are illustrated in the accompanying drawings and described below. While the invention will be described in conjunction with exemplary embodiments, it will be understood that present description is not intended to limit the invention to those exemplary embodiments.

### EXAMPLE 1

### Materials and Methods

1-1: Experimental Materials

Kumkang Woorimil species were obtained from National Institute of Crop Science (NICS), germinated and cultivated on a sterile organic peat moss for germination maintained at a constant temperature (average 20±2° C.). The Triticum aestivum Lamarck leaves cultivated for two weeks after germination were harvested, lyophilized, pulverized to a particular size and dried.

3T3-L1 preadipocytes were obtained from Korean Cell Bank Line (Seoul, Korea).

Dulbecco's modified Eagle's Media (DMEM) and fetal bovine serum (FBS) for cell culture were purchased from Hyclone (Logan, Utah, USA), and penicillin (100 unots/ml)-streptomycine (100 μg/ml) was purchased from WelGENE (Daegu, Korea). Cell Counting Kit-8 (CCK-8), a cell proliferation analysis reagent, was purchased from Dojindo (Tokyo, Japan); and insulin, dexamethasone and IBMX which were used for the differentiation of 3T3-L1 preadipocytes; and Oil Red O, which was used to confirm the formation of triglycerides within adipocytes, were purchased from Sigma-Aldrich (St. Luis, Mo., USA).

1-2: Preparation of Extracts

The i Triticum aestivum Lamarck leaves obtained in Example 1-1 were prepared into three different extracts (water, 50% ethanol, and 100% ethanol). The water extract was prepared by extracting 30 g of the lyophilized powder sample of Triticum aestivum Lamarck leaves using 1 L of purified water while heating, followed by filtration and concentration under reduced pressure using a rotary evaporator (N-1000, EYELA, Tokyo, Japan) to thereby obtain 3.2 g of water extract. 50% ethanol extract and 100% ethanol extract were prepared by extracting 30 g of the lyophilized powder sample of Triticum aestivum Lamarck leaves by extracting via sonication for one hour using 50% ethanol and 100% ethanol, respectively, and then concentration under reduced pressure using a rotary evaporator (N-1000, EYELA, Tokyo, Japan) to thereby respectively obtain 10 g and 4.5 g of extract therefrom. Among them, 10 g of the dried 50% ethanol extract was suspended in triple distilled water, and dichloromethane (CH2Cl2) (0.75 g), ethyl acetate (EtOAc)(3 g), and butanol (BuOH)(5 g) fractions were obtained thereform via systemic fractionation. To further the dryness, the extracts were put into lyophilization, and the thus obtained Triticum aestivum Lamarck leaves extract and its fractions thereof were stored at 4° C. being blocked from light prior to experiments.

1-3: 3T3-L1 Cell Culturing and Differentiation

The culture and maintenance of 3T3-L1 preadipocytes (American Type Culture Collection, Rockville, Md.) were performed such that when the cells were grown 60% in DMEM medium under 5% CO₂, 37° C. they were subcultured. The cells were detached and aliquoted into a 24-well plate with 2×10⁴ cells/well and cultured so that the cells become condensed 100%. 3T3-L1 Preadipocytes are known to be best maintained in such a condition. Two days after the 3T3-L1 cells reached a confluent stage, they were treated with a DMEM medium containing 10% FBS and MDI solution (0.5 mM IBMX, 0.1 μM dexamethasone, and 10 μg/mL of insulin) for 2 days, further treated with a DMEM medium containing 10% FBS and 10 μg/mL of insulin for 2 days, and then replaced with a DMEM medium containing 10% FBS and cultured for a total of 8 days, and the differentiation into adipocytes based on the formation of fat globules within the 3T3-L1 cells was confirmed.

1-4: Analysis of Cell Viability

The cytotoxicity of the 3T3-L1 preadipocytes by the extract was confirmed using a CCK-8 kit. The 3T3-L1 cells were aliquoted into a 96-well plate at a concentration of 5×10³cells/well and stabilized for 24 hours. The samples were treated with diluted media at varying concentrations, and cultured in an incubator set at 5% CO₂, 37° C. Then, 10 μL of the CCK-8 sample solution was added to each well, cultured for 1 hour, and the optical density of the culture was observed at 450 nm.

1-5: Oil Red O Staining

In order to confirm the generation of fat globules within the 3T3-L1 cells, the cells were dyed with Oil red O reagent which reacts with triglycerides. On the 8^{th} day from the differentiation, the culture medium was removed, washed with PBS buffer, and the cells were fixed with 4% formaldehyde at room temperature for 1 hour, and washed with 60% isopropanol. The resulting cells were dyed with Oil red O reagent for 20 minutes, washed with distilled water 3 times, and observed under a phase-contrast microscope. In addition, for quantitative analysis, fats were extracted from the cells using 100% isopropanol, and the resulting cells were transferred into a 96-well plate with a volume of 200 μL/well and their optical density was measured at 540 nm using ELISA reader. The content of the fat stained with Oil red O reagent was expressed as percentages relative to the average optical density of a control group.

1-6: Analysis of the Amount of Expression of FAS and PPAR-g

Western blot analysis was performed in order to confirm the expression of fatty acid synthetase (FAS) which is expressed in differentiated adipocytes. The 3T3-L1 cells were cultured in the presence of MDI solution for 48 hours and washed with PBS twice. Then, the resultant was added with PRO-PREP protein extraction solution (iNtRON Biotech., Gyeonggi-do, Korea) and dissolved at 4° C. for 20 minutes, centrifuged at 4° C. for 20 minutes to recover the supernatant and the proteins obtained thereof were quantitated. 30 μg of protein was collected from each cell as a specimen and mixed with SDS loading buffer, and electrophoresed by loading them into a 10% or 7.5% SDS polyamide gel, and the resulting gel was transferred onto a PVDF membrane. The membrane was blocked with a 5% skim milk solution to perform an antibody-antigen response. The antibody-antigen response was performed at 4° C. for 18 hours using rabbit antifatty acid synthase (FAS) mAb or rabbit anti-PPARg mAb (Cell Signaling, Danvers, Mass.) as a primary antibody. Then, the membrane was washed and reacted for 40 minutes with HRP-conjugated goat anti-rabbit IgG polyAb as a secondary antibody, and added with Chemiluminescent detection kit (Invitrogen Co., CA, USA) to develop a particular protein.

1-7: Handling Statistics

The experimental results were expresses as mean±standard deviation (SD), and statistical significance was treated after handling the difference between groups via Student's t-test, and evaluated with significance at the level of p<0.05.

### EXAMPLE 2

### Cytotoxiciy Test

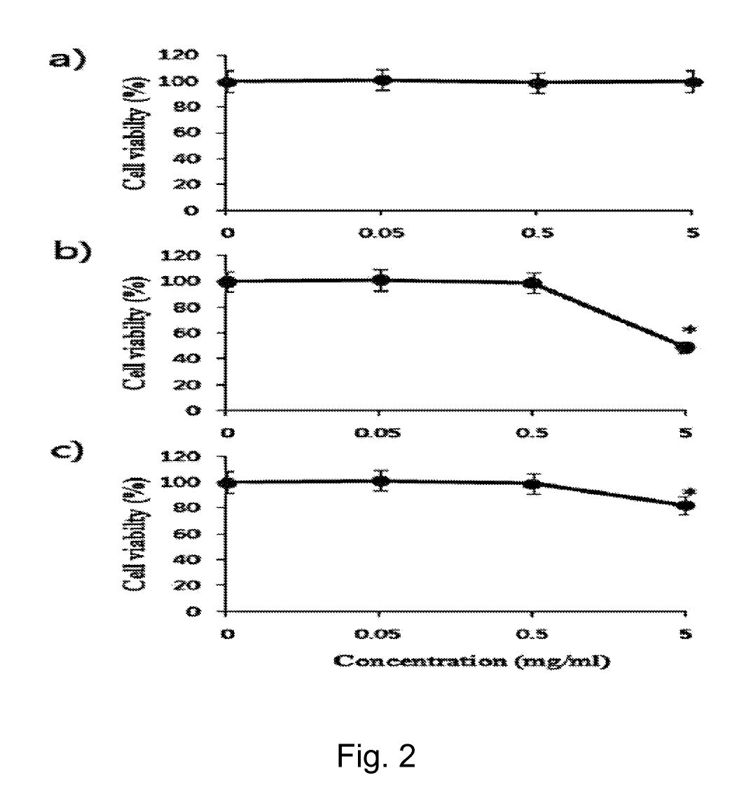

In order to confirm the cytotoxicity range of water extract, 50% ethanol extract, and 100% ethanol extract of Triticum aestivum Lamarck leaves using 3T3-L1 preadipocytes, cell viability was measured via a CCK-8 kit for analyzing cell proliferation (FIG. 2). The above extracts at concentrations of 0, 0.05, 0.5 and 5 mg/mL were respectively cultured with the 3T3-L1 cells for 48 hours and their cytotoxicity was measured. The result showed that the extracts did not affect on the viability of the 3T3-L1 cells up to the concentration of 0.5 mg/mL. Accordingly, in the subsequent experiments inducing differentiation of the 3T3-L1 preadipocytes, the concentration used was up to 0.5 mg/mL where no cytotoxicity was observed.

### EXAMPLE 3

### Effect of Inhibiting Fat Generation

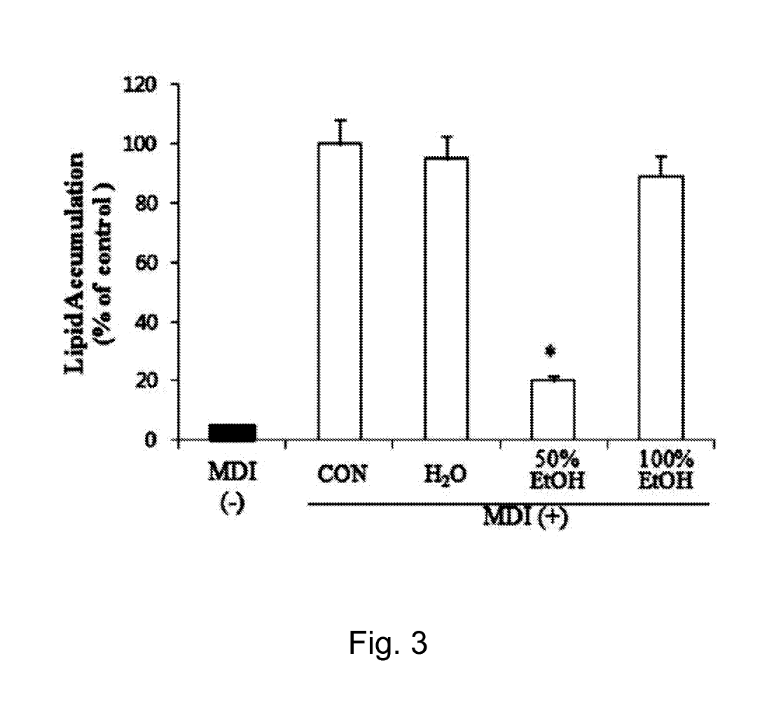

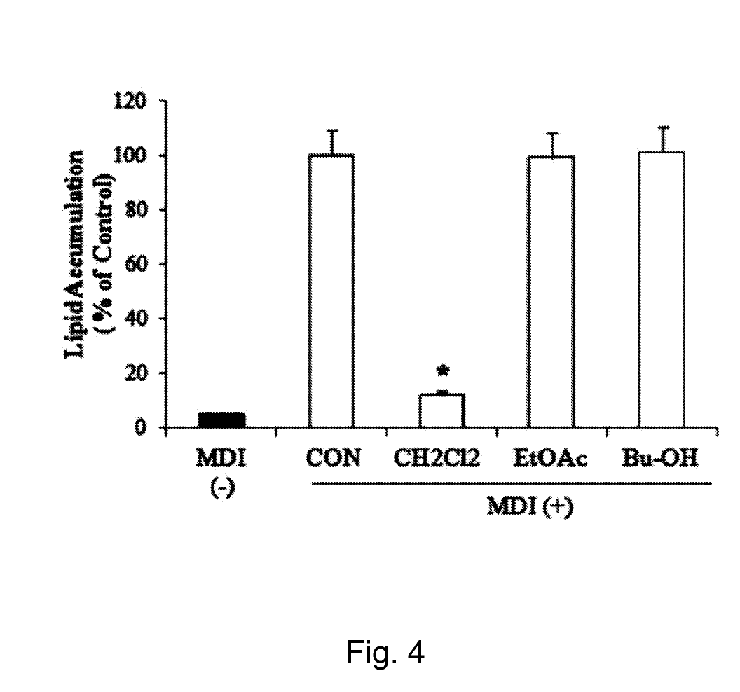

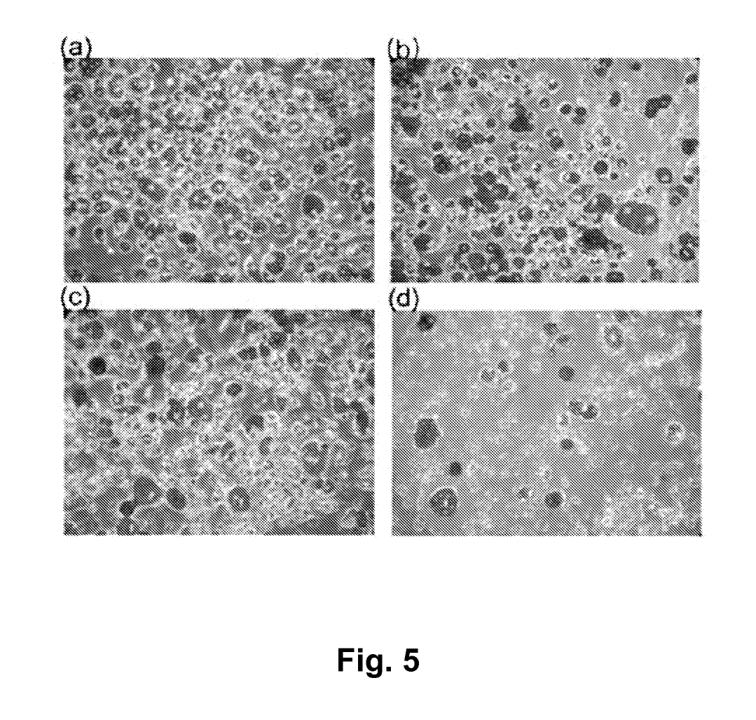

3T3-L1 preadipocytes were treated with water extract, 50% ethanol extract, and 100% ethanol extract of Triticum aestivum Lamarck leaves at a concentration of 0.5 mg/mL, and differentiation of the 3T3-L1 preadipocytes were induced according to an exemplary embodiment of Example 1-1, and dyed with Oil-Red O reagent in order to confirm the effect of the above extracts on the accumulation of fat globules and triglycerides within the 3T3-L1 cells. As a result, the formation of fat globules was observed only in the 3T3-L1 cells treated with only MDI differentiation inducing agent, and the accumulation of triglyceride was confirmed by dyeing with Oil-Red O reagent. As shown in FIG. 3, the 3T3-L1 cells were not affected in their differentiation into adipocytes when treated with water extract and 100% ethanol extract. However, the 3T3-L1 cells were affected in their differentiation into adipocytes when treated with 50% ethanol extract, and the content of triglyceride dyed with Oil-Red O reagent were substantially inhibited to the level undifferentiated cells. Accordingly, it was speculated that the 50% ethanol extract of Triticum aestivum Lamarck leaves includes an active material which can inhibit the differentiation of preadipocytes into adipocytes. In this regard, fractions obtained via systemic fractionation an alcohol extract of Triticum aestivum Lamarck leaves in the order of dichloromethane, ethyl acetate, and butanol, and the effect of each fraction on the differentiation of preadipocytes was investigated. First, in order to confirm the cytotoxicity of each fraction thereof, cell viability at concentrations of 0, 5, 50, 500 mg/mL was observed in the same manner as used for the 50% ethanol extract of Triticum aestivum Lamarck leaves. The result showed that ethyl acetate, and butanol fractions did not affect the cell viability at the highest concentration but the dichloromethane fraction did not affect up to the concentration of 50 mg/mL. Accordingly, in the experiment inducing differentiation of 3T3-L1 preadipocytes, the inhibitory effect of 50 mg/mL concentration, where no cytotoxicity was observed, against the differentiation into adipocytes was investigated (FIG. 4). As a result, the dichloromethane fraction at a concentration of 50 mg/mL derived from the 50% ethanol extract of Triticum aestivum Lamarck leaves was observed to be of significance in terms of inhibiting the differentiation of the treated 3T3-L1 cells into adipocytes. Additionally, in the cells whose differentiation was induced by MDI solution, the relative triglyceride content within the 3T3-L1 preadipocytes treated with the dichloromethane fraction showed an 88% decrease compared to that dyed with Oil Red O reagent in the 3T3-L1 cells induced by MDI solution. In contrast, ethyl acetate, and butanol fractions did not affect the cell viability. In addition, in the microscopic examination after dyeing with Oil-Red O reagent, it was confirmed that the dichloromethane fraction inhibited the accumulation of fats of the 3T3-L1 cells at concentrations of 0, 10, 25 or 50 mg/mL in a concentration-dependent manner (FIG. 5).

### EXAMPLE 4

### Effect of Inhibiting the Differentiation of Adipocytes

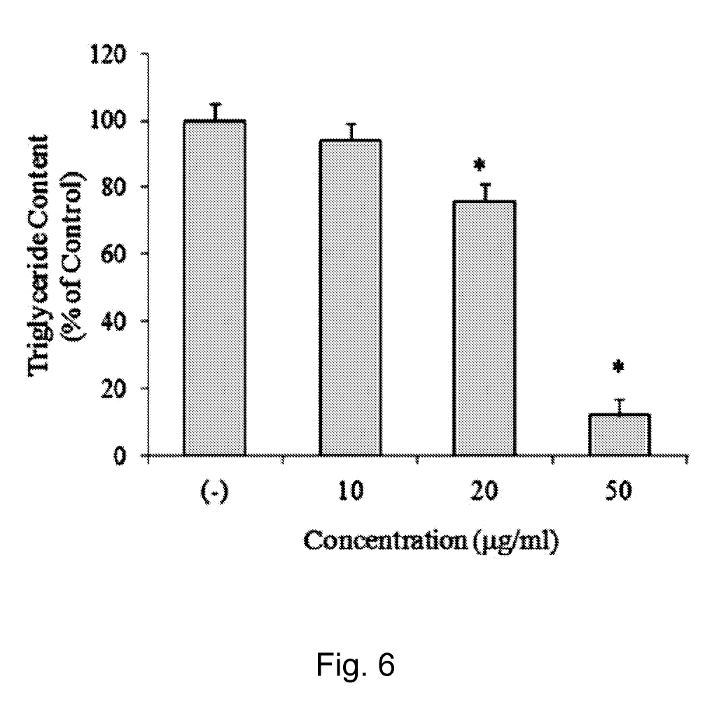

Triglycerides are continuously produced in the body due to metabolism of glucose being introduced into cells and denovo fatty acid synthesis process within cells, and accumulated in the fat globules, which bind to each other thereby increasing their size as time goes by, thereby resulting in the accumulation of triglycerides. Since fat globules in adipocytes consist of lipids such as triglycerides and perilipin A, proteins, etc., the content of triglycerides was measured via enzyme assay in order to more accurately confirm the decrease in the content of triglycerides (FIG. 6). As shown in the result of Oil Red O reagent dyeing method, the content of triglycerides in the differentiated 3T3-L1 cells was shown to decrease in a concentration-dependent manner.

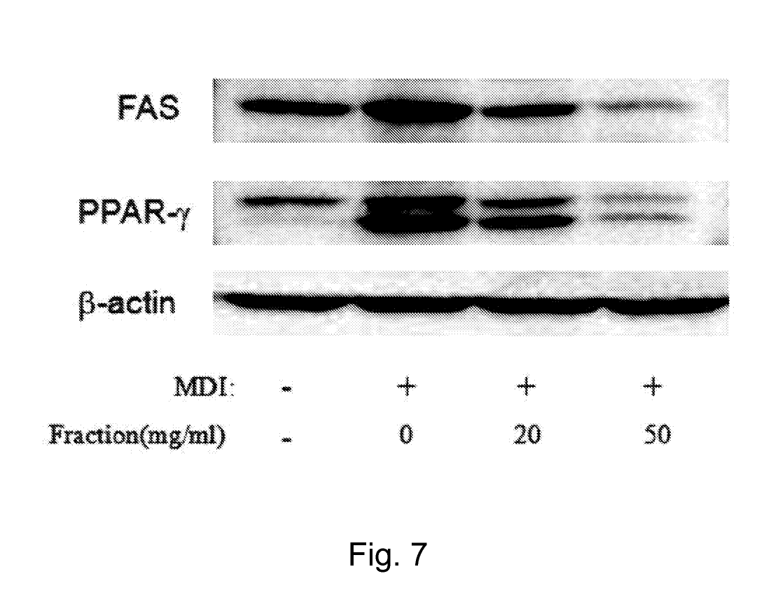

The first step of the differentiation of adipocytes is to induce the expression of transcription factors in response to insulin, which include the expression of C/EBPβ and C/EBPδ, and then C/EBPα and PPARγ. The responses of the transcription factors induce the synthesis of proteins involved in the differentiation of adipocytes, and stimulate the continuous expression of FAS, a triglyeride-synthesizing enzyme. The effect of the dichloromethane fraction derived from the 50% ethanol extract on the expression of proteins PPARγ and FAS during the differentiation of adipocytes was investigated via Western blot analysis (FIG. 7). When the 3T3-L1 preadipocytes were placed in the presence of MDI solution and 10% FBS for 2 days, and then in the presence of insulin and 10% FBS for 2 days, for a total of 4 days, and analyzed the expression level of PPARγ and FAS while they were differentiated, it was confirmed that the expression level of PPARγ and FAS considerably increased only in the cells which were treated with an MDI differentiation inducing agent. In addition, it was confirmed that the expression of PPARγ and FAS was inhibited in a concentration-dependent manner in the cells which were respectively treated with 20 μg/mL and 50 μg/mL of the dichloromethane fraction in the presence of an MDI solution. In particular, in the cells which were treated with 50 μg/mL of the dichloromethane fraction derived from the 50% ethanol extract, it was confirmed that the expression of PPARγ and FAS was inhibited to a level similar to that of the 3T3-L1 preadipocytes, i.e., a negative control. As such, it was very interesting that only the dichloromethane fraction derived from 50% ethanol extract of Triticum aestivum Lamarck leaves showed a considerable decrease in significance. There are two hypotheses available that may explain the reason why only the 50% ethanol extract inhibited the differentiation of the preadipocytes. First, it is speculated that active ingredients may be polar substances that are well dissolved both in water and alcohol. Second, the 100% ethanol fraction and the water fraction may contain substances that neutralize adipogenesis inhibitors or protects cells.

### PREPARATION EXAMPLE 1

### Manufacture of a Pharmaceutical Preparation

A pharmaceutical preparation including the dichloromethane fraction of the extract of Triticum aestivum Lamarck leaves was manufactured as follows.

1. Preparation of Powders

dichloromethane fraction of the extract of Triticum aestivum Lamarck leaves in Example 1 2 g

lactose 1 g

The above ingredients were mixed and loaded into an airtight sac to produce a powder agent.

2. Preparation of Tablets

dichloromethane fraction of the extract of Triticum aestivum Lamarck leaves in Example 1 100 mg

| | | |
|---|---|---|
| corn starch | 100 | mg |
| lactose | 100 | mg |
| magnesium stearate | 2 | mg |

These ingredients were mixed and prepared into tablets using a typical tabletting method.

3. Preparation of Capsules

dichloromethane fraction of the extract of Triticum aestivum Lamarck leaves in Example 1 100 mg

| | | |
|---|---|---|
| corn starch | 100 | mg |
| lactose | 100 | mg |
| magnesium stearate | 2 | mg |

These ingredients were mixed and loaded into gelatin capsules according to a typical method to produce capsules.

4. Preparation of Pills

dichloromethane fraction of the extract of Triticum aestivum Lamarck leaves in Example 1 1 g

| | | |
|---|---|---|
| lactose | 1.5 | g |
| glycerine | 1 | g |
| xylitol | 0.5 | g |

These ingredients were mixed and prepared into pills, each weighing 4 g, according to a conventional method.

5. Preparation of Granules

dichloromethane fraction of the extract of Triticum aestivum Lamarck leaves in Example 1 150 mg

| | | |
|---|---|---|
| soybean extract | 50 | mg |
| glucose | 200 | mg |
| starch | 600 | mg |

These ingredients were admixed together and 100 mg of 30% ethanol was added to the admixture. Drying at 60° C. gave granules which were then loaded into a sac.

### PREPARATION EXAMPLE 2

### Manufacture of Foods

Unmilled rice, barley, glutinous rice, and unshelled adlay were pregelatinized using a typical method, dried and roasted before grinding into powder with a particle size of 60 meshes. Black soybean, black sesame and wild sesame were steamed according to a typical method, dried and roasted before grinding into powder with a particle size of 60 meshes. The dichloromethane fraction of the extract of Triticum aestivum Lamarck leaves in Example 1 of the present invention was concentrated in a vacuum using a vacuum concentrator, sprayed, and dried in a convection oven, followed by grinding into powder with a particle size of 60 meshes.

The powders made of the grains, the seeds, and the extract according to the present invention were formulated at the following ratios to yield a food.

grains (30 wt % of unpolished rice, 15 wt % of adlay, and 20 wt % of barley),

seeds (7 wt % of perilla seeds, 8 wt % of black beans, and 7 wt % of black sesame),

dry powder of dichloromethane fraction of the extract of Triticum aestivum Lamarck leaves (3 wt %),

Lingshi mushroom (0.5 wt %),

Rehmannia root (0.5 wt %)

While exemplary embodiments of the present invention have been described in detail, the protection scope of the present invention is not limited to the foregoing embodiments and it will be appreciated by those skilled in the art that various substitutions, modifications and changes using the basic concept of the present invention defined in the appended claims are also included in the protection scope of the present invention.

1. A pharmaceutical composition for prevention or treatment of hyperlipidemia and fatty liver disease comprising a dichloromethane fraction as an active ingredient out of the fractions obtained by sequential systemic fractionation of an alcohol extract of Triticum aestivum Lamarck leaves with dichloromethane, ethyl acetate, and butanol, in that order.

2. The pharmaceutical composition according to claim 1, wherein the alcohol extract is a 50% ethanol extract.

3. The pharmaceutical composition according to claim 1, wherein the dichloromethane fraction inhibits expression of peroxisome proliferator-activated receptor-gamma (PPARγ) protein.

4. The pharmaceutical composition according to claim 1, wherein the dichloromethane fraction inhibits expression of fatty acid synthase (FAS) protein.

5. A health food composition for prevention or improvement of obesity comprising a dichloromethane fraction as an active ingredient out of the fractions obtained by sequential systemic fractionation of an alcohol extract of Triticum aestivum Lamarck leaves with dichloromethane, ethyl acetate, and butanol in that order.

6. The health food composition according to claim 5, wherein the alcohol extract is a 50% ethanol extract.

7. The health food composition according to claim 5, wherein the dichloromethane fraction inhibits expression of peroxisome proliferator-activated receptor-gamma (PPARγ) protein.

8. The health food composition according to claim 5, wherein the dichloromethane fraction inhibits expression of fatty acid synthase (FAS) protein.
